SEQUENCE LISTING (8 sequences)

SEQ ID NO: 1 - protein, 748 aa, Escherichia coli
MLTRLREIVEKVASAPRLNEALNILVTDICLAMDTEVCSVYLADHDRRCYYLMATRGLKKPRGRTVTLAFDEGIVGLVGRLAEPINLADAQKHPSFKYIPSVKEERFRAFLGVPIIQRRQLLGVLVVQQRELRQYDESEESFLVTLATQMAAILSQSQLTALFGQYRQTRIRALPAAPGVAIAEGWQDATLPLMEQVYQASTLDPALERERLTGALEEAANEFRRYSKRFAAGAQKETAAIFDLYSHLLSDTRLRRELFAEVDKGSVAEWAVKTVIEKFAEQFAALSDNYLKERAGDLRALGQRLLFHLDDANQGPNAWPERFILVADELSATTLAELPQDRLVGVVVRDGAANSHAAIMVRALGIPTVMGADIQPSVLHRRTLIVDGYRGELLVDPEPVLLQEYQRLISEEIELSRLAEDDVNLPAQLKSGERIKVMLNAGLSPEHEEKLGSRIDGIGLYRTEIPFMLQSGFPSEEEQVAQYQGMLQMFNDKPVTLRTLDVGADKQLPYMPISEENPCLGWRGIRITLDQPEIFLIQVRAMLRANAATGNLNILLPMVTSLDEVDEARRLIERAGREVEEMIGYEIPKPRIGIMLEVPSMVFMLPHLAKRVDFISVGTNDLTQYILAVDRNNTRVANIYDSLHPAMLRALAMIAREAEIHGIDLRLCGEMAGDPMCVAILIGLGYRHLSMNGRSVARAKYLLRRIDYAEAENLAQRSLEAQLATEVRHQVAAFMERRGMGGLIRGGL

SEQ ID NO: 2 - protein, 163 aa, Escherichia coli
MTNNDTTLQLSSVLNRECTRSRVHCQSKKRALEIISELAAKQLSLPPQVVFEAILTREKMGSTGIGNGIAIPHGKLEEDTLRAVGVFVQLETPIAFDAIDNQPVDLLFALLVPADQTKTHLHTLSLVAKRLADKTICRRLRAAQSDEELYQIITDTEGTPDEA

SEQ ID NO: 3 - protein, 90 aa, Escherichia coli
MTVKQTVEITNKLGMHARPAMKLFELMQGFDAEVLLRNDEGTEAEANSVIALLMLDSAKGRQIEVEATGPQEEEALAAVIALFNSGFDED

SEQ ID NO: 4 - protein, 323 aa, Escherichia coli
MTIAIVIGTHGWAAEQLLKTAEMLLGEQENVGWIDFVPGENAETLIEKYNAQLAKLDTTKGVLFLVDTWGGSPFNAASRIVVDKEHYEVIAGVNIPMLVETLMARDDDPSFDELVALAVETGREGVKALKAKPVEKAAPAPAAAAPKAAPTPAKPMGPNDYMVIGLARIDDRLIHGQVATRWTKETNVSRIIVVSDEVAADTVRKTLLTQVAPPGVTAHVVDVAKMIRVYNNPKYAGERVMLLFTNPTDVERLVEGGVKITSVNVGGMAFRQGKTQVNNAVSVDEKDIEAFKKLNARGIELEVRKVSTDPKLKMMDLISKIDK

SEQ ID NO: 5 - DNA, 34 bp, synthetic construct
  misc_feature 1..34: note = Primer
  misc_feature 20..29: note = n is a, c, g, or t
  source 1..34: mol_type = other DNA
gccacgcgtcgactagtacnnnnnnnnnnacgcc

SEQ ID NO: 6 - DNA, 19 bp, synthetic construct
  misc_feature 1..19: note = Primer
  source 1..19: mol_type = other DNA
gccacgcgtcgactagtac

SEQ ID NO: 7 - DNA, 23 bp, synthetic construct
  misc_feature 1..23: note = Primer
  source 1..23: mol_type = other DNA
catgatcctctagagtcgacctg

SEQ ID NO: 8 - DNA, 18 bp, synthetic construct
  misc_feature 1..18: note = Primer
  source 1..18: mol_type = other DNA
caggcatgcaagcttcag

(19) United States
(12) Patent Application Publication
(10) Pub. No.: US 2025/0059259 A1
(43) Pub. Date: Feb. 20, 2025

(54) KNOCKOUT OF ptsP GENE ELEVATES ACTIVE GENE EXPRESSION

(71) Applicant: ABSCI CORPORATION, Vancouver, WA (US)

(72) Inventor: Matthew Weinstock, Vancouver, WA (US)

(21) Appl. No.: 18/724,015

(22) PCT Filed: Dec. 22, 2022
(86) PCT No.: PCT/US2022/082294

§ 371 Date: Jun. 25, 2024

(51) Int. Cl. C07K 16/00; C12N 15/70

(52) U.S. Cl. CPC C07K 16/00; C12N 15/70; C12N 2800/101; C12N 2830/002

(57) ABSTRACT

Variant or mutant prokaryotic cells are disclosed that express elevated or increased levels of a wide variety of active proteins. Also disclosed are methods of producing the elevated or increased levels of active encoded proteins.

### CROSS-REFERENCE TO RELATED APPLICATION

This application claims priority to U.S. Provisional Application No. 63/295,278, filed Dec. 30, 2021, which is incorporated by reference herein in its entirety.

### FIELD

The disclosed subject matter relates generally to the field of active protein expression in cells and, more particularly, to the elevated expression of active protein in prokaryotic mutant host cells such as mutated Escherichia coli.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “57386_Seqlisting.XML”, which was created on Dec. 7, 2022 and is 10,989 bytes in size. The subject matter of the Sequence Listing is incorporated herein by reference in its entirety.

### BACKGROUND

Organisms (e.g., E. coli) used for bioproduction of therapeutic proteins evolved to thrive in their natural environmental niches (e.g., the dynamic environment of mammalian gut), not for the production of proteins in a controlled laboratory environment. As such, it is a reasonable hypothesis that E. coli contains endogenous genes that are important in their natural environmental niche, but that limit or negatively impact the organism's ability to produce recombinant proteins in unnatural environments, such as in the lab or in commercial-scale bioreactor facilities. Given that there are about 5000 genes in E. coli, evaluating the impact of the removal of each of these endogenous genes on recombinant protein expression poses a significant technical challenge.

Transposons are mobile genetic elements that can randomly insert into the genome of an organism, potentially disrupting (thereby deactivating) genes at the insertion site. Transposons can be harnessed in a process known as transposon mutagenesis to generate a library of bacterial cells where each member of the population contains a random gene disruption due to a unique transposon insertion. If the library size is large enough, the majority of non-essential genes (those genes not required for survival in the laboratory environment) are anticipated to be disrupted in the library. With a library of strains mutagenized in this manner, the challenge is to 1) construct a screening process to identify strains in the library that contain a gene disruption that improves recombinant protein production, 2) develop a method to determine which gene has been disrupted in the strains with increased protein production, and 3) make a “clean” deletion (i.e., reconstruct the deletion in the background strain using strain engineering techniques). The most challenging step is step 1 (constructing a suitable screening process). Transposons have been used in the manner described above in metabolic engineering campaigns where the screens are simple, such as visual inspection of colonies on a plate to identify colonies that are visually distinct (e.g., brighter color in the case of carotenoid biosynthesis). While it would be easy to apply this approach to the expression of certain kinds of proteins (e.g., fluorescent proteins where one could isolate mutant cells that have higher protein expression by using fluorescence), applying this approach to therapeutic proteins that don't have an easily measurable signal is non-trivial due to the dearth of high-throughput screens capable of measuring expression/activity of therapeutic proteins.

### SUMMARY

Disclosed is a process by which gene knockout libraries were created and screened to determine the impacts of specific gene knockouts on active protein expression levels. The results of the experimental work disclosed herein led to the discovery that a defect in a prokaryotic phosphotransferase system (i.e., PTS), as exemplified by a knockout of the ptsP gene (SEQ ID NO:1), resulted in stably elevated titers of active gene expression for a group of genes encoding diverse proteins. The increased level of active protein is expressed in a variety of host cells, such as E. coli host cells engineered to have a more oxidizing cytoplasmic environment than wild-type E. coli.

In one aspect, the disclosure provides a prokaryotic organism comprising a defective PhosphoTransfer System (i.e., PtsP pathway) and a heterologous expressible coding region or gene. In some embodiments, the prokaryotic organism comprises a defective PtsP pathway. In some embodiments, the prokaryotic organism comprises a defective PTS operon, such as by comprising a defective ptsl, ptsO (SEQ ID NO:3), ptsN (SEQ ID NO:2) ptsP (SEQ ID NO:1), or manX (i.e., ptsL; SEQ ID NO:4) coding region or gene. In some embodiments, the prokaryotic organism comprises a defective ptsP gene (SEQ ID NO:1) coding region. In some embodiments, the heterologous expressible coding region or gene is located on an episome, and in other embodiments in the chromosome of the prokaryotic organism. In some embodiments, the organism is derived from an Enterobacterial species, such as Escherichia coli. In some embodiments, the organism comprises a ptsP⁻ coding region or gene having at least a partial deletion of the sequence set forth in SEQ ID NO:1. In some embodiments, the ptsP⁻ coding region or gene comprises a deletion of no more than 10 contiguous nucleotides of SEQ ID NO:1. In some embodiments, the ptsP⁻ coding region or gene comprises at least one missense or nonsense mutation compared to the sequence set forth in SEQ ID NO:1. In some embodiments, the prokaryotic organism comprises an intracellular environment that is more oxidizing than a wild-type prokaryotic organism of the same species. In some embodiments, the prokaryotic organism comprises the ahpC^{Δ} allele of the gene encoding peroxiredoxin and an inactivating mutation of (a) trxB encoding thioredoxin, (b) gor encoding glutaredoxin reductase, or (c) both trxB and gor. In some embodiments, the prokaryotic organism further comprises at least one inactivating mutation in the araBAD operon. In some embodiments, the prokaryotic organism further comprises ΔaraEp::J23104. In some embodiments, the prokaryotic organism further comprises λatt::pNEB3-r1-cDsbC (Spec, lacl). In some embodiments, the prokaryotic organism further comprises ΔscpA-argK-scpBC. In some embodiments, the prokaryotic organism comprises ahpC^{Δ}, trxB⁻, gor⁻, ΔscpA-argK-scpBC, and at least one inactivating mutation in the araBAD operon.

The disclosure further provides embodiments wherein the episome is a viral vector, plasmid, phagemid, or artificial chromosome. In some embodiments, the prokaryotic organism further comprises ΔaraEp::J23104. In some embodiments, the ΔaraEp::J23104 is located on an episome. In some embodiments, the prokaryotic organism further comprises an inducible promoter in operable linkage to the expressible coding region or gene, such as wherein the inducible promoter and the expressible coding region or gene are located on an episome. In some embodiments, the inducible promoter is ParaBAD. In some embodiments, the ParaBAD promoter is located on an episome. In some embodiments, the prokaryotic organism further comprises ParaC in operable linkage to araC, PprpR in operable linkage to prpR, and ParaBAD in operable linkage to the expressible coding region or gene. In some embodiments, the ParaC, araC, PprpR, prpR, ParaBAD, and the expressible coding region or gene are located on an episome. In some embodiments, the prokaryotic organism further comprises at least one chaperone coding region or gene. In some embodiments, the chaperone coding region or gene is located on an episome. In some embodiments, the expressible coding region or gene encodes an antibody chain, an antibody fragment thereof, or a cytokine.

Another aspect of the disclosure is drawn to a method comprising: (a) introducing an episome comprising an expressible coding region or gene into a prokaryotic host cell comprising a defective PtsP pathway; and (b) culturing the prokaryotic host cell under conditions compatible with expression of the coding region or gene; wherein the level of expression of active encoded product from the heterologous expressible coding region or gene is greater than the level of expression of active encoded product from the coding region or gene in a wild-type counterpart of the prokaryotic host cell. A “wild-type” strain is the E. coli counterpart strain to the E. coli mutant or variant strain having a defect in the PtsP pathway (i.e., the same E. coli strain as the strain with the PtsP pathway defect but without the PtsP pathway defect). Unless otherwise stated, the wild-type strain is the ptsP⁺ counterpart to the ptsP⁻ E. coli strain disclosed in the Examples and throughout this application. In the methods according to the disclosure, a greater level of expression of active encoded product is achieved by increasing the titer of expressed product or by increasing the production of properly folded, soluble, active product, or both. In some embodiments, the level of expression of active encoded product is at least 2%, 3%, 4%, 5%, 6%, 7%, 8%, 9%, 10%, 11%, 12%, 13%, 14%, 15%, 18%, 20%, 22%, 25%, 28%, 30%, 35%, 40%, 45%, 50%, 55%, 60%, 65%, 70%, 75%, 80%, 85%, 90%, 95% 100%, 110%, 120%, 130%, 140%, 150%, 160%, 170%, 180%, 190%, 200%, 250%, 300%, 350%, 400% 450%, 500%, 600%, 700%, 800%, 900%, 1000% or greater in the prokaryotic host cell than in the wild-type counterpart of the prokaryotic host cell. In some embodiments, the greater level of expression of active encoded product is due to an increased percentage of expressed product that is in an active conformation. In some embodiments, the prokaryotic host cell is derived from an Enterobacterial species, such as Escherichia coli. In some embodiments, the prokaryotic host cell comprises a ptsP⁻ coding region or gene. In some embodiments, the ptsP⁻ coding region or gene comprises at least a partial deletion of the sequence set forth in SEQ ID NO:1. In some embodiments, the ptsP⁻ coding region or gene comprises a deletion of no more than 10 contiguous nucleotides of SEQ ID NO:1. In some embodiments, the ptsP⁻ coding region or gene comprises at least one missense or nonsense mutation compared to the sequence set forth in SEQ ID NO:1.

In other embodiments, the prokaryotic organism comprises an intracellular environment that is more oxidizing than a wild-type prokaryotic organism of the same species. In some embodiments, the prokaryotic organism comprises the ahpC^{Δ} allele of the gene encoding peroxiredoxin and an inactivating mutation of trxB encoding thioredoxin, an inactivating mutation of gor encoding glutaredoxin reductase, or inactivating mutations of both genes. In some embodiments, the prokaryotic organism comprises at least one inactivating mutation in the araBAD operon. In some embodiments, the prokaryotic organism further comprises λatt::pNEB3-r1-cDsbC (Spec, lacl). In some embodiments, the prokaryotic organism further comprises ΔscpA-argK-scpBC. In some embodiments, the prokaryotic organism comprises ahpC^{Δ}, trxB⁻, gor⁻, ΔscpA-argK-scpBC, and at least one inactivating mutation in the araBAD operon. In some embodiments, the episome is a viral vector, plasmid, phagemid, or artificial chromosome. In some embodiments, the prokaryotic organism comprises ΔaraEp::J23104. In some embodiments, the episome further comprises an inducible promoter in operable linkage to the expressible coding region or gene. In some embodiments, the inducible promoter is ParaBAD. In some embodiments, the episome comprises ParaC in operable linkage to araC, PprpR in operable linkage to prpR, and ParaBAD in operable linkage to the expressible coding region or gene. In some embodiments, the episome further comprises at least one chaperone coding region or gene in operable linkage to PprpB. In some embodiments, the expressible coding region or gene encodes an antibody chain, an antibody fragment thereof, or a chimeric antigen receptor.

Other features and advantages of the disclosure will be better understood by reference to the following detailed description, including the examples.

### BRIEF DESCRIPTION OF THE DRAWINGS

FIG. 1. Growth study of ptsP⁻ E. coli strain compared to ptsP⁺ wild-type E. coli control strain. The start of expression induction is indicated by the arrows. Reflectance is measured as a function of elapsed culture time.

FIG. 2. Average heterodimer recovery in grams per liter as a function of time from ptsP⁻ knockout E. coli strain and from its control ptsP⁺ E. coli strain.

FIG. 3. Graphs of mean active heterodimer protein 1 (Fab 1) levels expressed over time from an expression construct in various E. coli mutant strains resulting from Tn5 mutagenesis.

FIG. 4. A histogram showing the HiPrBind assay results of two-factor binding of active heterodimeric gene product in ptsP⁻ knockout E. coli strain and in control ptsP⁺ E. coli strain.

FIG. 5. Heterodimer titer of Fab 2 active gene product in ptsP⁻ E. coli compared to the positive control strain over time.

FIG. 6. A histogram showing the results of a two-factor HiPrBind assay of active expressed protein 5 (Bi-specific antibody) heterodimer in E. coli strains comprising the expression construct for protein 5.

### DETAILED DESCRIPTION

Disclosed herein is the transposon mutagenesis of a population of prokaryotic cells, such as proteobacterial cells as exemplified by γ-proteobacterial cells such as Escherichia coli. Mutagenized cells were cultivated, protein expression was induced, and Activity-specific Cell Enrichment (i.e., ACE) assays were performed to provide a basis for sorting the population of cells into bins based on the level of active protein expression, thereby allowing for isolation and identification of top expressors. The sites at which the transposon inserted into the genome in this population of ACE-enriched cells was recovered via a semi-arbitrary PCR process. Genomic DNAs were sequenced using next generation sequencing (i.e., Illumina's MiSeq deep sequencing instrument), and an analysis pipeline was developed to count the number of times each gene in the genome was disrupted in the population. Gene knockouts that were highly enriched in this population were identified, and two-factor HiPrBind assays confirmed the elevated levels of active gene product expression. The top 18 most enriched gene knockouts were reconstructed in a clean E. coli strain engineered to have a semi-oxidizing cytoplasmic environment conducive to active protein expression. These strains were tested for expression of active proteins of interest introduced into the cells using expression constructs to determine the impact on active protein expression of the various gene knockouts prevalent in the enriched cell population. One knockout (ptsP) stood out as dramatically increasing the titer of a variety of active gene products, as disclosed in the Examples.

### The Pts Pathway

Bacteria have intricate regulatory networks to coordinate carbon and nitrogen metabolism. Central components of these regulatory networks are the regulatory phosphotransferase systems (PTSs). In bacteria, e.g., gram-negative bacteria, there are two common PTS pathways: the carbohydrate-PTS, which coordinates carbohydrate transport, and the nitrogen-related PTS (PTS^{Ntr}), a signal transduction cascade with various regulatory roles. Proteobacteria, a diverse phylum of gram-negative bacteria, typically implement an integrated PTS comprising (1) PTS^{Ntr}, encoded by the genes ptsP (EI^{Ntr}), ptsO (Npr), ptsN (EIIA^{Ntr}), and ptsl, and (2) an EIIA component remaining from the carbohydrate-PTS, manX (EIIA^{Man}). The manX gene is also known as ptsL. The PTS^{Ntr} is present in a wide variety of α-, β-, and γ-proteobacteria. Although not wishing to be bound by theory, PtsP contains a GAF domain (a domain binding allosteric regulatory molecules) that may lead to variant or mutated prokaryotic cells of the disclosure when inactivated. In E. coli, glutamine and α-ketoglutarate control phosphorylation of PTS^{Ntr} through allosteric binding to the GAF domain of PtsP. PtsP acquires high-energy phosphate from phosphoenolpyruvate (PEP), which then phosphorylates the small carrier protein NPr (or HPr in the carbohydrate-PTS) on a conserved histidine residue. The disclosure provides prokaryotic organisms, such as E. coli, that comprise at least one inactivating gene or coding region mutation in the integrated PhosphoTransferase System or PTS. The disclosure expressly embraces prokaryotic organisms with a mutation in at least one of ptsP, ptsO, ptsN, ptsl, or manX (i.e., ptsL) that reduces at least one activity of the encoded gene product by at least 80%. In certain embodiments, the variant or mutated prokaryotic cells comprise at least one gene or coding region of the PTS^{Ntr}, e.g., ptsP, ptsO, ptsN, or ptsl that exhibits at least an 80% reduction in at least one activity. In certain embodiments, the manX (ptsL) gene is mutated, with a resultant decrease of at least 80% in at least one activity of the encoded gene product.

### Host Cells

Host cells, such as prokaryotic host cells, are provided that comprise expression constructs designed for the expression of coding regions, including coding regions for fusion proteins. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, i.e., proteobacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

As described in WO/2017/106583, incorporated by reference herein in its entirety, producing gene products such as therapeutic proteins at commercial scale and in soluble form is addressed by providing suitable host cells capable of growth at high cell density in fermentation culture, and which can produce soluble gene products in the oxidizing host cell cytoplasm through highly controlled inducible gene expression. Host cells of the present disclosure with these qualities are produced by combining some or all of the following characteristics. (1) The host cells are genetically modified to have an oxidizing cytoplasm by increasing the expression or function of oxidizing polypeptides in the cytoplasm, and/or by decreasing the expression or function of reducing polypeptides in the cytoplasm. Specific examples of such genetic alterations are provided herein and in WO 2017/106583. Optionally, host cells can also be genetically modified to express chaperones and/or cofactors that assist in the production of the desired gene product(s), and/or to glycosylate polypeptide gene products. (2) The host cells comprise one or more expression constructs designed for the expression of one or more active gene products of interest. In certain embodiments, at least one expression construct comprises an inducible promoter and a polynucleotide encoding a gene product to be expressed in active form from the inducible promoter. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is araE, araE, araG, araH, rhaT, xylF, xylG, or xylH, or particularly the transporter protein is araE, or wherein the alteration of gene function more particularly is expression of unaltered araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is scpA/sbm, argK/ygfD, scpB/ygfG, scpC/ygfH, rmlA, rmlB, rmlC, or rmlD.

Host Cells with Oxidizing Cytoplasm. The expression systems of the present disclosure are designed to express active gene products. In certain embodiments of the disclosure, the active gene products are expressed in a host cell. Examples of host cells are provided that allow for the efficient and cost-effective expression of active gene products, including components of multimeric products. In certain embodiments of the disclosure, the host cells are microbial cells such as gram-negative bacteria, e.g., E. coli. Exemplary E. coli host cells having oxidizing cytoplasm include the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H). The E. coli B strains with oxidizing cytoplasm are able to grow to much higher cell densities than the most closely corresponding E. coli K strain (WO/2017/106583).

Alterations to host cell gene functions. Certain alterations can be made to the gene functions of host cells comprising inducible expression constructs, to promote efficient and homogeneous induction of the host cell population by an inducer. Preferably, the combination of expression constructs, host cell genotype, and induction conditions results in at least 75% (more preferably at least 85%, and most preferably, at least 95%) of the cells in the culture expressing active gene product from each induced promoter, as measured by the method of Khlebnikov et al. described in Example 9 of WO/2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the coding region of the gene in its entirety, or by deleting a large enough portion of the gene, inserting sequence into the gene, or otherwise altering the gene sequence so that a reduced level of functional gene product is made from that gene, as is described herein with greater particularity for the ptsP gene or coding region. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of chaperone proteins, alterations of the reduction-oxidation environment of the host cell are also contemplated.

Host cell reduction-oxidation environment. In bacterial cells such as E. coli, proteins that need disulfide bonds to be active are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 10:32 (2011)). It is also possible to express cytoplasmic forms of these Dsb proteins, such as a cytoplasmic version of DsbA (cDsbA) and/or of DsbC (cDsbC), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in proteins, including heterologous proteins, produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with a defective thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (such as ahpC* and ahpCA, Lobstein et al., Microb Cell Fact 11:56 (2012)) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dupl62-169 (Faulkner et al., Proc Natl Acad Sci USA 105 (18):6735-6740 (2008), Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase I) catalase-peroxidase encoded by E. coli katG and HPII (hydroperoxidase II) catalase-peroxidase encoded by E. coli katE, which disproportionate peroxide into water and 0₂ (Farr and Kogoma, Microbiol Rev. 55 (4):561-585; (1991)). Increasing levels of KatG and/or KatE protein in host cells through induced co-expression or through elevated levels of constitutive expression is an aspect of some embodiments of the disclosure.

The disclosure also contemplates the expression of the sulfhydryl oxidase Ervlp, derived from the inner membrane space of yeast mitochondria, in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 10:1 (2011)).

Host cells comprising expression constructs preferably also express cDsbA and/or cDsbC and/or Ervlp, are deficient in trxB gene function, and are also deficient in the gene function of either gor, gshB, or gshA. Optionally, the host cells have increased levels of katG and/or katE gene function, and express an appropriate mutant form of AhpC so that the host cells can be grown in the absence of dithiothreitol (i.e., DTT).

Cellular transport of cofactors. When using the expression systems of the disclosure to produce enzymes that require cofactors for function, it is helpful to use a host cell either capable of synthesizing the cofactor from available precursors, or capable of taking it up from the environment. Common cofactors include ATP, coenzyme A, flavin adenine dinucleotide (FAD), NAD⁺/NADH, and heme. Polynucleotides encoding cofactor transport polypeptides and/or cofactor synthesizing polypeptides can be introduced into host cells, and such polypeptides can be constitutively expressed, or inducibly co-expressed with the active gene products to be produced by methods of the disclosure.

Proteases. Host cells can have alterations in their ability to degrade expressed protein products because of the lack of or lowering of the activity of one or more proteases. Exemplary protease include, but are not limited to, Clp, ClpP, OmpT, Lon, FtsH, ClpX, ClpY, ClpA, ClpQ, ClpAP, ClpXP, ClpAXP, ClpYQ, ClpY, and the proteases encoded by yaeL, sppA, tldD, sprT, yhbU, ptrA, frvX, hyaD, hybD, hycH, envC, ddpX, degP, degQ, degS, hslV, hslU, pepB, pepP, sohB, yggG, pepE, pepN, pepQ, abgA, pepT, iadA, pepA, pepD, ptrB, ycaL, ycbZ, yegQ, ygeY, ypdF, hycI, sgcX, and htpX.

Glycosylation of polypeptide gene products. Host cells can have alterations in their ability to glycosylate polypeptides. For example, eukaryotic host cells can have eliminated or reduced gene function of the glycosyltransferase and/or oligo-saccharyltransferase genes, impairing the normal eukaryotic glycosylation of polypeptides to form glycoproteins. Prokaryotic host cells such as E. coli, which do not normally glycosylate polypeptides, can be altered to express a set of eukaryotic and prokaryotic genes that provide a glycosylation function (DeLisa et al., WO 2009/089154A2).

### Transposon Mutagenesis

For transposon-mediated mutagenesis, a commercially available form of Tn5, i.e., the EZ-Tn5™ Transposome, was introduced into an E. coli B strain under conditions suitable for Tn5 mobilization, as is known in the art. Subsequently, cells exposed to Tn5 were cultured under selection for markers borne by EZ-Tn5™ (e.g., erm^{r}, amp^{r}). This protocol is standard in the field and led to random insertions of Tn5 into non-essential genes throughout the E. coli genome.

Expression Constructs. In some embodiments of the disclosure, inducible promoters are contemplated for use with the expression constructs to be introduced into the host cells according to the disclosure in order to achieve elevated expression of desired active gene products. Exemplary promoters are described herein and are also described in WO/2016/205570, incorporated herein by reference in relevant part. As described herein, the cells comprising one or more expression constructs may optionally include one or more inducible promoters to express a gene product of interest. In one embodiment, the gene product is a fusion protein. In other embodiments, the gene product is a protein, for example a therapeutic protein.

Expression constructs are polynucleotides designed for the expression of one or more gene products of interest, and thus are not naturally occurring molecules. Any expression construct known in the art is contemplated for use in the cells and methods of the disclosure, including expression constructs that can be integrated into a host cell chromosome or maintained within the host cell as extra-chromosomal, independently replicating polynucleotide molecules, i.e., episomes having origins of replication independent of the host cell chromosome, such as plasmids or artificial chromosomes. Expression constructs according to the disclosure also may have one or more selectable markers to enable selection of those cells harboring the expression construct. Exemplary selectable markers confer resistance to antibiotics lethal to the host cell lacking that selectable marker or encode enzymes required to produce essential nutrients. Any selectable marker known in the art is contemplated for use in the expression constructs of the disclosure. Expression markers may also contain an inducible promoter to provide the ability to induce the expression of a coding region operably linked to that inducible promoter. Exemplary inducible promoters contemplated by the disclosure include the arabinose promoter (ParaBAD), ParaC, ParaE, the propionate promoter (PprpBCDE), the rhamnose promoter (PrhaSR), the xylose promoter (PxylA), the lactose promoter, and the alkaline phosphatase promoter. Additional information on contemplated inducible promoters, including the sequences thereof, is provided in WO 2016/205570, incorporated herein by reference in relevant part. In addition to inducible promoters, the disclosure comprehends expression constructs comprising constitutive promoters. To ensure that RNA transcribed from the expression construct is efficiently translated, the construct may also include a ribosome binding site (RBS). In prokaryotes in general (archaea and bacteria), the RBS consensus sequence is GGAGG or GGAGGU, and in bacteria such as E. coli, the RBS consensus sequence is further defined as AGGAGG or AGGAGGU. To facilitate incorporation of a coding region or gene of interest, the expression construct may include a multiple cloning site in which a variety of restriction endonuclease cleavage sites are clustered to provide flexibility in incorporating exogenous polynucleotides, as is known in the art. Some embodiments of the expression construct of the disclosure further include a coding region for a signal peptide or leader peptide, wherein the coding region is oriented to result in expression of fusion protein comprising the signal peptide and the active gene product of interest.

### Assays: Activity-Specific Cell-Enrichment (ACE) Assay and HiPrBind Assay

The activity-specific cell-enrichment (ACE) assay identifies host cells that express active gene product of interest rather than inactive material, as described in WO 2021/146626, incorporated herein in relevant part. Active gene product can be distinguished from inactive material by the ability of active gene product to specifically bind a binding partner molecule, or by the ability of gene product to participate in a chemical or enzymatic reaction, as examples. The presence of properly formed disulfide bonds in a polypeptide gene product is an indication that it is correctly folded and presumptively active. In the cell-enrichment methods, active gene product of interest is detected by utilizing an appropriate labeling complex that specifically binds to active gene product of interest, such as a labeled antigen if the gene product of interest is an antibody or Fab; or a labeled ligand if the gene product of interest is a receptor or a receptor fragment, where the ligand specifically binds to an active conformation of the receptor; or a labeled substrate or a labeled substrate analog if the gene product of interest is an enzyme, as examples. For any gene product of interest, if there is an available antibody or antibody fragment that specifically binds to the active gene product and not to inactive gene product, that antibody or antibody fragment can be used to label the active gene product of interest when attached to a detectable moiety.

The HiPrBind assay provides an efficient method for multiple interrogations of an active gene product, such as by providing at least two distinct interrogations of a characteristic property of the active gene product or simultaneously interrogating at least two characteristic properties of that active gene product. HiPrBind assays are described in WO 2021/163349, incorporated herein in relevant part. The assay is an advance on the principle underlying the yeast two hybrid assay in that a multi-component detection mechanism is brought into proximity, and thereby brought into an environment where the detection mechanism can be active in producing a signal capable of detection. One component of the multi-component (e.g., two component) detection system is stably associated with a first analyte-associating moiety (i.e., active gene product-associating moiety) and a second component of the detection system is stably associated with a distinct second analyte-associating moiety. A detectable signal is generated when the two components of the detection system are brought into proximity by the analyte-associating moieties binding to the analyte. Because each of the analyte-associating moieties is specific for an active gene product as analyte, a signal is only generated when a characteristic property of an active gene product is detected using two distinct mechanisms, or when two distinct characteristic properties of an active gene product are simultaneously detected. The HiPrBind assay is versatile in detecting a variety of characteristic properties, but a simple example involves a gene product that is active in homodimeric form wherein each monomer requires disulfide bonds to properly fold. One active gene product-associating moiety can be a binding agent that specifically binds to the properly folded and therefore active monomer, and a second active gene product-associating moiety can be a distinct second binding agent that specifically binds to the dimeric form of the gene product. Thus, the HiPrBind assay in this embodiment simultaneously detects a gene product that is properly folded and in dimeric form.

### Chaperones

In some embodiments, an active gene product may be co-expressed with another gene product, such as a chaperone, that facilitates formation of the active form of the gene product of interest. Chaperones are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome. Exemplary chaperones present in E. coli host cells are the folding factors Dnak/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. Dnak/DnaJ/GrpE, GroEL/GroES, and ClpB can function synergistically in assisting protein folding, and expression of these chaperones in various combinations has been shown to facilitate expression of properly folded gene product. When expressing eukaryotic proteins in prokaryotic host cells, a eukaryotic chaperone protein, such as protein disulfide isomerase (PDI) from the same or a related eukaryotic species, can be co-expressed, e.g., inducibly co-expressed, with the gene product of interest.

### Strain Construction and Testing

The nucleic acid of cell variants exhibiting high levels of active gene product expression is amplified using semi-arbitrary PCR and the amplified fragments are subjected to next generation sequencing, yielding an ordered frequency of Tn5 insertion sites. This information was then used to construct variant strains with the identified mutation in stable form in a clean genotypic background. Typically, the gene or coding region insertionally inactivated by a transposon in the initial screen for cells supporting elevated expression of active gene product are deleted to create a stable mutation. By constructing such strains, the mutation giving rise to the advantageous elevated expression levels was stabilized, in part by having a mutation independent of a mobile genetic element such as a transposon, and freed from any unknown additional variation in the genome.

Inactivating gene mutations of the prokaryotic organisms of the disclosure reduce at least one activity of the encoded gene product by at least 80% relative to wild-type, such as a mutation that completely eliminates at least one activity of the encoded gene product. One form of mutation of a PTS gene or coding region (e.g., ptsP, ptsO, ptsN or manX) compatible with the variant or mutant prokaryotic organisms of the disclosure is insertional inactivation. As noted above, insertional inactivation using a mobile genetic element such as a transposon is valuable in generating mutants to be screened, but is not preferred in constructing stably mutagenized strains once the genetic element(s) to be mutagenized have been identified. Any stable insertion that inactivates or yields an 80% reduction in at least one activity of a PTS gene product is contemplated by the disclosure, as is a stable deletion of 1, 2, 3, 4, 5, 6, 7, 8, 9, 10, 15, 20, 30, 40, 50 or more nucleotides of a PTS gene or coding region. The disclosure further extends to single nucleotide substitutions (i.e., point mutations) or clustered mutations of 2, 3, 4, 5, 6, 7, 8, 9, 10 or more nucleotides that inactivate or reduce by at least 80% at least one activity of a PTS gene or coding region. One or more single nucleotide substitutions, whether clustered or not, are contemplated, both within the coding region of a PTS protein and outside the coding region in a polynucleotide region that affects the control of expression. As would be understood in the art, single nucleotide substitutions in the first or second position of codons would be more likely to result in non-silent mutations than variations at the third position of codons, but the disclosure contemplates nucleotide substitutions in all positions within the coding region that alter the encoded amino acid sequence and result in an expressed gene product with at least one activity reduced by at least 80%. Single nucleotide substitutions according to the disclosure may be transitions (either purine to purine or pyrimidine to pyrimidine) or transversions (purine to pyrimidine and pyrimidine to purine). Further contemplated are frameshift mutations in the coding region of a PTS protein that inactivate or reduce at least one activity by at least 80% relative to wild-type levels. It is also contemplated that the phenotype of elevated expression of active gene product can be induced dynamically using any of the various forms of RNAi engineered for inducible expression of the RNAi, or using inducible expression of antisense RNA.

### EXAMPLES

### Example 1

### Transposon Mutagenesis

Global transposon mutagenesis was performed by introducing Transposon 5 (Tn5) via the EZ-Tn5™ Transposome into an E. coli strain using techniques standard in the art. Following Tn5 mutagenesis, cells were cultured on plates under tetracycline antibiotic selection to obtain cells harboring Tn5. Vidal et al., PLOS ONE 4 (7):e6232 (2009). The random insertion of Tn5 into the E. coli genome followed by culturing the mutagenized cells under standard conditions on selection media generated a random population of non-essential gene disruptions by Tn5 insertional inactivation of the genes.

### Example 2

### Cell Culture

The resultant mutagenized E. coli strains of Example 1 were cultured under standard scalable culture conditions known in the art.

### Example 3

### ACE Assays, HiPrBind Assays, and Expression Binning

The ACE assay, disclosed herein and in WO 2021/146626, incorporated herein by reference in relevant part, allows for the interrogation of very large libraries and the sorting of the libraries based on the amount of, or activity level of, active proteins of potential or known therapeutic value.

The activity-specific cell-enrichment methods identify host cells that express active gene product of interest rather than inactive material. Active gene product can be distinguished from inactive material by the ability of active gene product to specifically bind a binding partner molecule, or by the ability of gene product to participate in a chemical or enzymatic reaction, as examples. The presence of properly formed disulfide bonds in a polypeptide gene product is an indication that it is correctly folded and presumptively active. In the cell-enrichment methods, active gene product of interest is detected by utilizing an appropriate labeling complex that specifically binds to active gene product of interest, such as a labeled antigen if the gene product of interest is an antibody or Fab; or a labeled ligand if the gene product of interest is a receptor or a receptor fragment, where the ligand specifically binds to an active conformation of the receptor; or a labeled substrate or a labeled substrate analog if the gene product of interest is an enzyme, as examples. For any gene product of interest, if there is an available antibody or antibody fragment that specifically binds to the active gene product and not to inactive gene product, that antibody or antibody fragment can be used to label the active gene product of interest when attached to a detectable moiety.

Genetic diversity in a population of host cells can result, for example, from genomic variation among the host cells, induced, for example, by transposon mutagenesis (e.g., Tn5 mutagenesis). Genetic diversity can also result from differences in the polynucleotide sequences of expression constructs induced, for example, by transposon mutagenesis such as Tn5 mutagenesis, and the host cells disclosed herein can comprise such genetically diverse polynucleotides. If there is genomic diversity among the host cells, due to chromosomal diversity resulting, e.g., from transposon mutagenesis, selecting high-performing host cells and sequencing genomic DNA recovered from them can be used to identify genomic differences, such as mutations, associated with the superior performance of the selected host cells. If there is diversity among expression constructs in the host cell population, recovering the expression constructs, such as expression vectors, from the selected host cells and sequencing the expression constructs can permit creation of a library of expression constructs (a ‘high-performance library’) that comprises those expression construct elements associated with high-performing host cells. A population of live high-performing host cells can be reconstructed by transforming a parental host cell strain with the high-performance library, or with the recovered high-performing expression constructs themselves. A parental host cell strain can be the strain used to create the host cell population that was screened for high-performing host cells, or another strain that can be genetically modified or transformed with expression constructs to create a host cell strain capable of expressing the gene product of interest.

The activity-specific cell-enrichment methods involve the following aspects: (1) providing a genetically diverse population of host cells that can comprise expression constructs; (2) labeling the gene product of interest within the host cells by expressing the gene product of interest as a detectable fusion protein, or by contacting the gene product of interest with a labeling complex that specifically binds the active gene product of interest; (3) selecting high-performing host cells using a sorting apparatus that employs flow cytometry or a comparable method; (4) analyzing the selected host cells and/or expression vectors; (5) reconstructing host cell strains; (6) optionally further analyzing reconstructed host cell strains, particularly with respect to the activity of the gene product of interest; and (7) optionally repeating any or all of steps (1)-(6) above.

The activity-specific cell-enrichment methods provided take full advantage of the flow cytometer's speed of sample analysis to fractionate or bin E. coli variants based on the level of expression of active gene product, resulting in the isolation of high-performing host cells, such as those that express more active gene product of interest. In some embodiments, populations of host cells over one million in diversity can be analyzed within minutes to determine whether a higher-performing subset population exists. If so, and if the flow cytometer is a FACS instrument, several hundred higher-performing host cells from a rare (one in one million) subpopulation can be isolated within an hour to enable subsequent analysis. The criteria that define subpopulations of host cells can include none, some, or all the host cells of the population within the defined subpopulation; in some instances, the subpopulation may be coextensive with the population. For example, a subpopulation of host cells, defined by expression of a labeled gene product of interest at levels detectable by a flow cytometer, can include all-or a substantial majority of-the host cells of the population.

The cultures of Example 2 were subjected to Activity-specific Cell Enrichment (ACE) assays to identify cells expressing the plasmid-borne coding region at the highest level of active protein.

To confirm that strains exhibiting elevated expression of active gene product did stably express such levels of active gene product, mutagenized cells were subjected to HiPrBind assays, which are engineered to yield a signal only when two independent interrogations of an active protein of interest are successful. As noted above, one component of the two-component detection system is stably associated with a first analyte-associating moiety (i.e., active gene product-associating moiety) and a second component of the detection system is stably associated with a distinct second analyte-associating moiety. A detectable signal is generated when the two components of the detection system are brought into proximity by the analyte-associating moieties binding to the analyte, which is an active protein of interest. In one embodiment, the active protein of interest was an active Fab.

### Example 4

### PCR Amplification and Sequencing

A population of high-producing cells was subjected to polymerase chain reaction amplification using a two-stage, nested, semi-arbitrary PCR process in which one PCR primer from each stage annealed to the 3′ end of Tn5 and the other primer was a degenerate mixture of sequences. The Tn5-specific primer used in stage 1 annealed in outward orientation within 150 nucleotides of the Tn5 terminus, and the Tn5-specific primer used in stage 2 annealed closer to the Tn5 terminus than the primer for stage 1, thereby resulting in nested amplification of the terminal sequence of Tn5 fused to E. coli genomic DNA at the site of Tn5 insertion. The other member of the PCR primer pair used for each of stages 1 and 2 was a degenerate mixture of primer sequences that enabled this mix of PCR primers to hybridize throughout the E. coli genome. At least one of the primers in the mix would hybridize in the proper orientation and proper distance from the PCR primer specific for Tn5 leading to amplified nucleic acid fragments in each of the two stages, with the Tn5 sequence included in the amplified PCR product available for identification of the precise point of Tn5 insertion into the E. coli genome. PCR primers used in the experiments disclosed herein included Primer 1 (5′-GCCACGCGTCGACTAGTACNNNNNNNNNNACGCC-3′; SEQ ID NO:5), Primer 2 (5′-GCCACGCGTCGACTAGTAC-3′; SEQ ID NO:6), Primer 3 (5′-CATGATCCTCTAGAGTCGACCTG-3′; SEQ ID NO:7), and Primer 4 (5′-CAGGCATGCAAGCTTCAG3′; SEQ ID NO:8). Such amplified nucleic acid fragments were subjected to rapid, highly parallel next generation sequencing using an Illumina MiSeq deep sequencing instrument with standard Nextera Flex sample preparation. Alternative approaches to next generation sequencing, e.g., use of amplicon sequencing kits, alternative NGS apparatus, are also available and contemplated for use in methods according to the disclosure. Sequencing results were analyzed to identify Tn5 insertion site sequences in the E. coli genome and to order them by frequency of occurrence.

### Example 5

### Insertion Site Mapping

The semi-arbitrary PCR amplification process yielded fusion sequences containing Tn5 sequences abutting host cell genomic sequences. The known Tn5 sequences facilitated identification of the E. coli genomic sequences, which were then compared to the known sequence of the E. coli chromosome. An annotated version of the E. coli chromosomal DNA sequence facilitated identification of disrupted E. coli genes. The insertion site mapping data, when coupled with the insertion site frequency data, yielded a frequency order of E. coli genes facilitating increased expression of active gene product. Those mutated genes with the highest frequencies of increased expression of active gene product were selected for further analysis to determine the range of genes whose expressed active products would be increased relative to controls such as wild-type E. coli active gene product expression.

### Example 6

### Strain Construction and Scale-Up

The top 18 gene disruptions in terms of expressed active protein levels were reconstructed to stabilize the mutations and eliminate subsequent genetic modification due to Tn movement. Strain construction involved isolation and identification of the mutated DNA, typically a gene, and re-construction of the mutation in a stable structural form that is free of the Tn5 mobile genetic element used to initially generate the mutation. The results of the experimental work disclosed in Examples 1-5 led to the identification of E. coli ptsP as a gene that, when inactivated by mutation, led to dramatic and wide-spread increases in the expression of plasmid-borne coding regions, including genes. It is expected that a variation or mutation in any of the genes of the PhosphoTransfer System, i.e., ptsP, ptsN, ptsO, ptsl, or manX, that reduces at least one activity of the encoded gene product by at least 80% will yield a prokaryotic host cell according to the disclosure that exhibits elevated expression levels for a wide variety of active gene products.

### ptsP⁻ E. coli Strain

The E. coli strain containing random transposon integrations was transformed with an expression construct for Fab 1 and induced in both large and small bioreactors. Samples were sorted by ACE assay and binned according to expression level using FACS sorting and labeled binding partners specific for the expressed active gene product. DNA was extracted from the high-expressor gate. The DNA was subjected to a semi-arbitrary PCR protocol to amplify the junction of the transposon insertion site in the genome. The DNA was sequenced on the MiSeq apparatus and reads were mapped to the transposon. For reads that mapped to the 3′ end of the transposon, the 20 bp downstream of the transposon (corresponding to the insertion site) were mapped to the genome using Geneious Prime bioinformatics software (Genious).

The results of the strain generation process (transposon mutagenesis, cell growth, screening of cells via ACE assay coupled to FACS sorting for binning, next generation sequencing, mutation mapping, HiPrBind assay confirmation of elevated active gene product expression, and strain construction) led to the development of multiple ptsP⁻ E. coli strains of various genetic backgrounds. In addition to the ptsP⁻ knockout mutation, these strains have been modified to create a more oxidized intracellular environment compared to wild-type E. coli. As a result, these strains express a markedly elevated level or increased titer of active protein for a wide variety of target proteins, including relatively large proteins such as the wide variety of antibody forms and chimeric antigen receptors (CARs).

### Example 7

### Elevated Expression of Protein 1 (Fab 1)

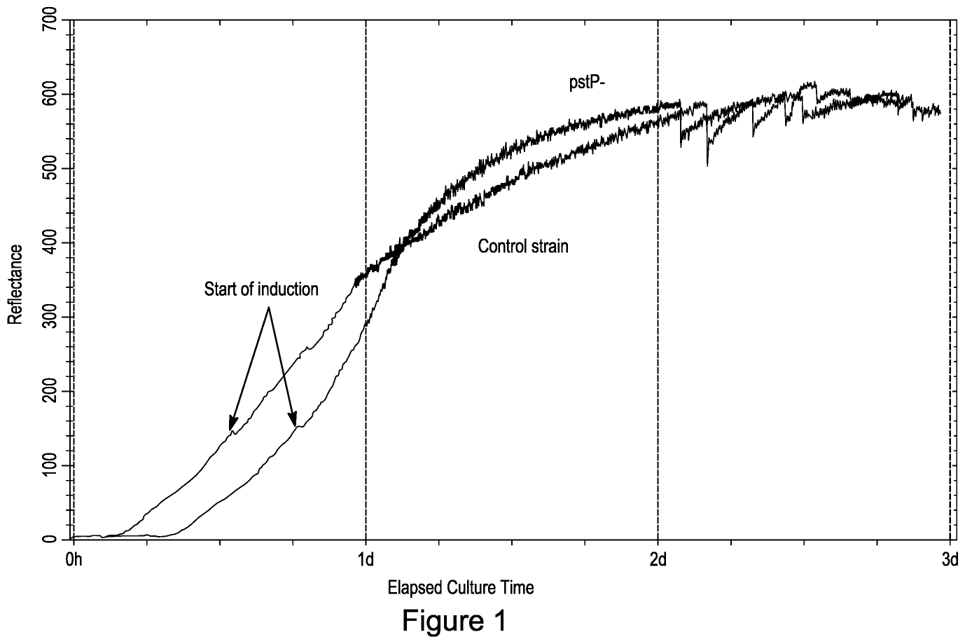

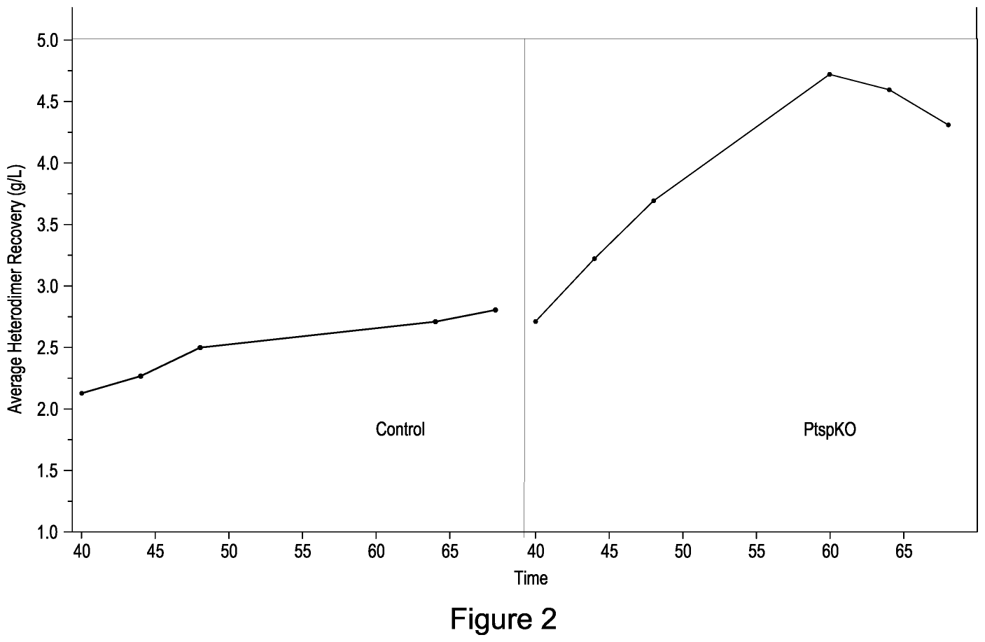

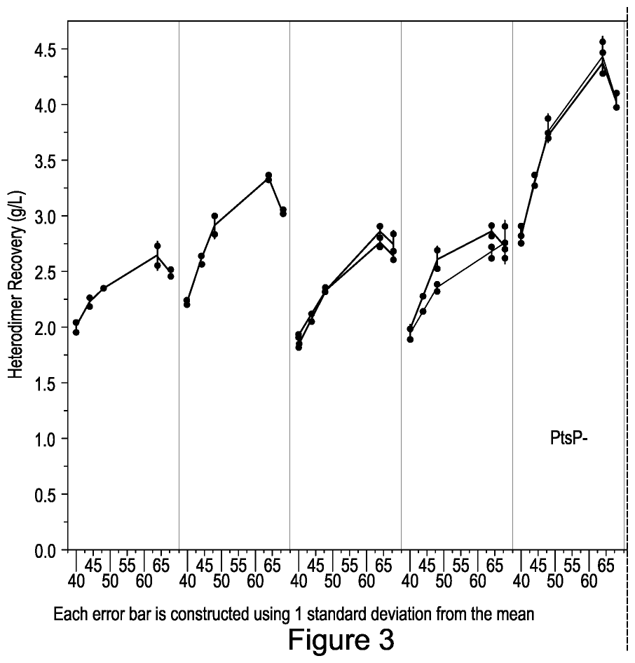

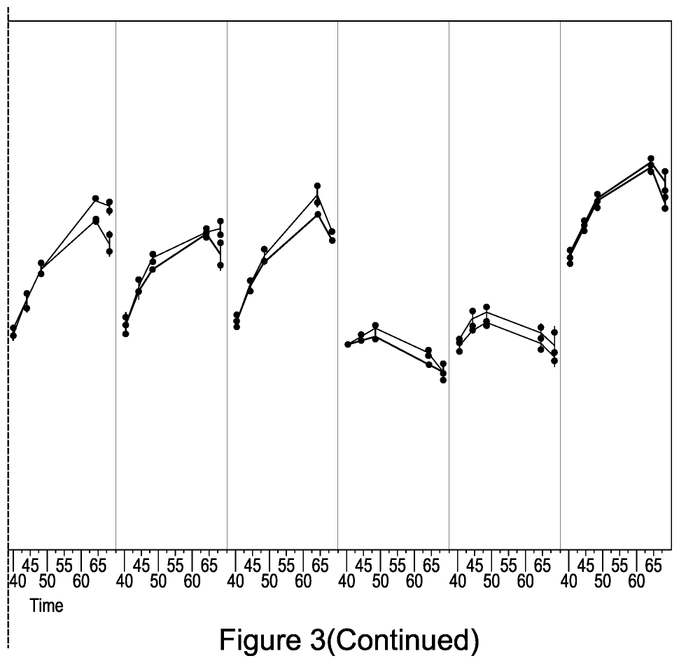

A ptsP⁻ strain of E. coli re-constructed as set forth in Example 6 and containing an expression construct encoding a Fab (i.e., Fab 1) was assessed for the relative and absolute levels of expression of Fab 1. As shown in FIG. 1, the ptsP⁻ strain grew in culture with similar dynamics to the background strain.

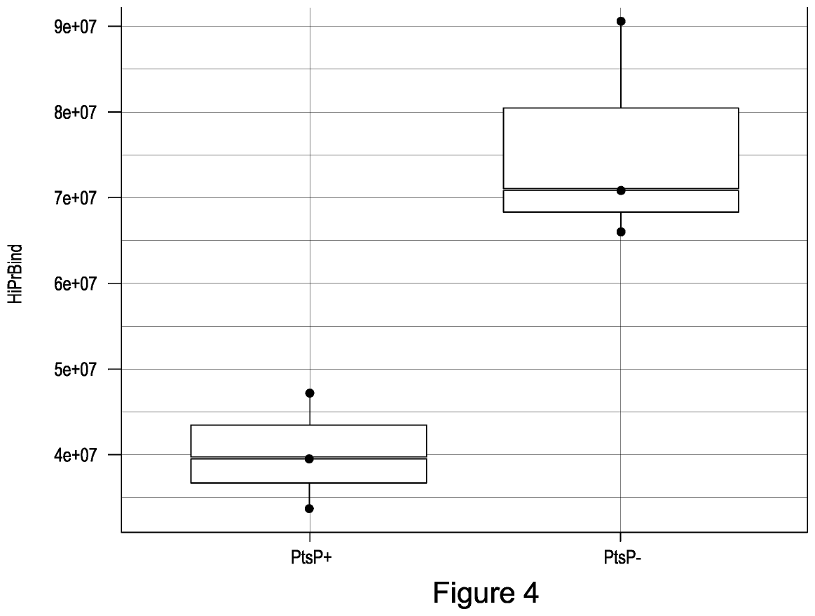

Additional expression studies confirmed the elevated levels of active gene product expression in multiple ptsP⁻ E. coli strains relative to control strain expression levels. FIG. 4 shows elevated average levels of recovered active Fab 1 expressed in the ptsP⁻ E. coli relative to the control strain.

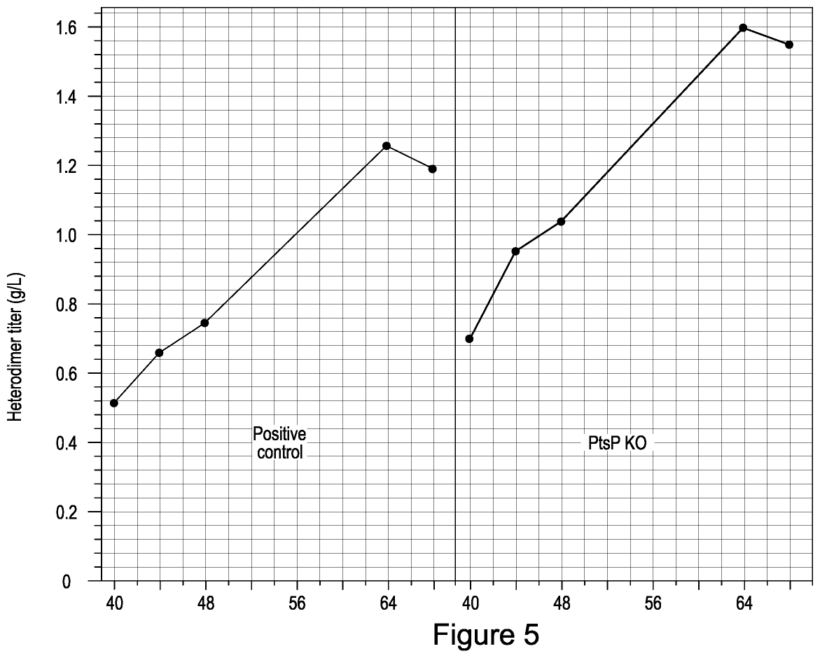

Levels of recovered active protein 1 (Fab 1) expressed in a variety of E. coli strains resulting from In mutagenesis were measured over time to assess expression levels in various genetic backgrounds. Expression levels of about 10 E. coli mutants comprising the expression construct for protein 1 were graphed as a function of culture time (FIG. 5). The results showed that the ptsP⁻ strains of exhibited markedly higher levels of active protein 1 gene product being expressed at all times relative to all other strains examined. HiPrBind assays of these strains yielded consistent data in that the ptsP⁻ strains of E. coli expressed elevated levels of active protein 1. Focusing on the effect of ptsP⁻ inactivation, HiPrBind assays of active protein 1 heterodimer expressed by the E. coli ptsP⁻ strain and by the E. coli ptsP⁺ strain showed that the E. coli ptsP⁻ strain expressed a markedly elevated level of active protein 1 heterodimer compared to the level expressed by the E. coli ptsP⁺ strain (FIG. 4).

### Example 8

### Elevated Expression of Protein 2 (Fab 2)

To determine if ptsP⁻ E. coli would produce elevated levels of an active gene product other than the Fab 1 of Example 7, a second, distinct Fab coding region (i.e., Fab 2) was cloned into an expression construct according to the disclosure. An expression construct comprising the coding region for Fab 2 was introduced into a control ptsP⁺ E. coli strain and into a ptsP⁻ E. coli strain. The ptsP⁻ E. coli strain expressed an elevated level of active Fab 2 gene product heterodimer, which was recovered and measured as a function of elapsed culture time, when compared to the level of Fab 2 expressed from the control strain E. coli (FIG. 5). Moreover, the ptsP⁻ E. coli strain expressed an elevated level of Fab 2 from the expression construct, relative to control, at all tested time points during culturing (FIG. 5).

### Example 9

### Elevated Expression of Additional Proteins

To determine if the elevated protein expression in a proteobacterium with an inactivated PhosphoTransfer System (PTS) would extend to gene products beyond the Fab molecules of protein 1 and protein 2, expression constructs encoding a variety of protein types were introduced into matched pairs of E. coli strains that were ptsP⁺ or ptsP⁻. HiPrBind assays of E. coli strains revealed that inactivation of ptsP alone elevated the expression of active protein 4. Unlike the Fab molecules of proteins 1 and 2, active protein 4 is a structural protein rich in α-helices and thus not similar to the β-sheet-based immunoglobulins in structure, which demonstrates that inactivation of the PhosphoTransfer System results in elevated expression of a wide variety of expressed proteins.

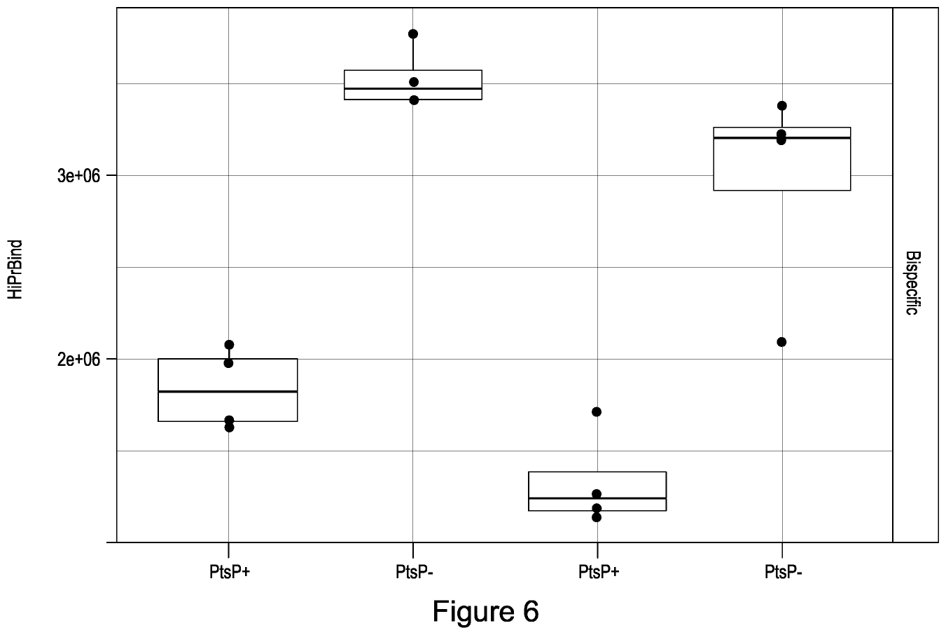

HiPrBind assays were also conducted on E. coli strains of various genetic backgrounds that comprised an expression construct for protein 5 (Bi-specific antibody). Results presented in FIG. 6 showed that both ptsP⁻ strains of E. coli demonstrated elevated expression of the Bi-specific antibody compared to the levels achieved with their respective control ptsP⁺ strains.

Strains comprising an expression construct for protein 6 (Fc fusion protein 1) were also subjected to HiPrBind assays to assess expression levels of active gene product. E. coli (ptsP⁻) produced markedly elevated levels of active protein 6 compared to E. coli (ptsP⁺ ).

Yet another HiPrBind study was conducted on E. coli strains of varying genetic backgrounds that comprised an expression construct for protein 8 (antibody fusion). The results showed that the ptsP⁻ E. coli expressed a significantly elevated level of protein 8 compared to ptsP⁺ E. coli strains of differing genetic backgrounds. These results are consistent with all of the results presented herein from widely varying protein types that consistently showed that inactivation of the PhosphoTransfer System, for example by inactivating ptsP, led to significantly elevated levels of active gene product being expressed.

All publications and patents mentioned in the application are herein incorporated by reference in their entireties or in relevant part, as would be apparent from context. Various modifications and variations of the disclosed subject matter will be apparent to those skilled in the art without departing from the scope and spirit of the disclosure. Although the disclosure has been described in connection with specific preferred embodiments, it should be understood that the invention as claimed should not be unduly limited to such specific embodiments. Various modifications of the described modes for making or using the disclosed subject matter that are obvious to those skilled in the relevant field(s) are intended to be within the scope of the following claims.

1. A prokaryotic organism comprising a defective PtsP pathway and a heterologous expressible coding region or gene.

2. The prokaryotic organism according to claim 1 comprising a defective PTS operon.

3. The prokaryotic organism according to claim 2 comprising a defective ptsl, ptsO, ptsN, ptsP, or manX gene.

4. (canceled)

5. (canceled)

6. The prokaryotic organism according to claim 1 wherein the heterologous expressible coding region or gene is located on an episome.

7. The prokaryotic organism according to claim 1 wherein the heterologous expressible coding region or gene is located in the chromosome of the prokaryotic organism.

8. (canceled)

9. (canceled)

10. The prokaryotic organism according to claim 1 wherein the organism comprises a ptsP⁻ coding region or gene.

11. The prokaryotic organism according to claim 10 wherein the ptsP⁻ coding region or gene comprises at least a partial deletion of the sequence set forth in SEQ ID NO:1.

12. The prokaryotic organism according to claim 11 wherein the ptsP⁻ coding region or gene comprises a deletion of no more than 10 contiguous nucleotides of SEQ ID NO:1.

13. The prokaryotic organism according to claim 10 wherein the ptsP⁻ coding region or gene comprises at least one missense or nonsense mutation compared to the sequence set forth in SEQ ID NO:1.

14. The prokaryotic organism according to claim 1 wherein the prokaryotic organism comprises an intracellular environment that is more oxidizing than a wild-type prokaryotic organism of the same species.

15. The prokaryotic organism according to claim 14 comprising the ahpC^{Δ} allele of the gene encoding peroxiredoxin and an inactivating mutation of
(a) trxB encoding thioredoxin,
(b) gor encoding glutaredoxin reductase, or
(c) both trxB and gor.

16.-19. (canceled)

20. The prokaryotic organism according to claim 1 comprising ahpC^{Δ}, trxB⁻, gor⁻, ΔscpA-argK-scpBC, and at least one inactivating mutation in the araBAD operon.

21. (canceled)

22. The prokaryotic organism according to claim 1 further comprising ΔaraEp::J23104.

23. (canceled)

24. The prokaryotic organism according to claim 1 further comprising an inducible promoter in operable linkage to the expressible coding region or gene.

25. The prokaryotic organism according to claim 24 wherein the inducible promoter and the expressible coding region or gene are located on an episome.

26. The prokaryotic organism according to claim 24 wherein the inducible promoter is ParaBAD.

27. (canceled)

28. The prokaryotic organism according to claim 1 further comprising ParaC in operable linkage to araC, PprpR in operable linkage to prpR, and ParaBAD in operable linkage to the expressible coding region or gene.

29. (canceled)

30. The prokaryotic organism according to claim 1 further comprising at least one chaperone coding region or gene.

31. (canceled)

32. The prokaryotic organism according to claim 1 wherein the expressible coding region or gene encodes an antibody chain, an antibody fragment thereof, or a chimeric antigen receptor.

33. A method comprising:
(a) introducing an episome comprising an expressible coding region or gene into a prokaryotic host cell comprising a defective PtsP pathway; and
(b) culturing the prokaryotic host cell under conditions compatible with expression of the coding region or gene;
wherein the level of expression of active encoded product from the heterologous expressible coding region or gene is greater than the level of expression of active encoded product from the coding region or gene in a wild-type counterpart of the prokaryotic host cell.

34.-54. (canceled)
